(19) United States
(12) Patent Application Publication
(10) Pub. No.: US 2019/0105219 A1
(43) Pub. Date: Apr. 11, 2019

(54) LEG SUPPORT APPARATUS

(71) Applicant: Dennis Pierro, Bloomfield, NJ (US)

(72) Inventor: Dennis Pierro, Bloomfield, NJ (US)

(21) Appl. No.: 16/153,060

(22) Filed: Oct. 5, 2018

Related U.S. Application Data
(60) Provisional application No. 62/568,512, filed on Oct. 5, 2017.

(51) Int. Cl. A61H 3/02; A61F 5/01; A61F 5/37

(52) U.S. Cl. CPC A61H 3/02; A61H 2003/007; A61F 5/3761; A61F 5/0106

(57) ABSTRACT

Systems and apparatuses for coupling a leg portion of a user to a crutch are provided. The system includes a crutch and a leg brace coupled to the crutch. The leg brace includes an adjustable height portion, a leg receiving portion and a securing mechanism configured to secure a portion of a leg of a user to the leg receiving portion.

### CLAIM OF PRIORITY

This application is a United States non-provisional application and claims priority to U.S. Provisional Patent No. 62/568,512, filed Oct. 5, 2017, herein incorporated by reference in its entirety.

### FIELD OF THE EMBODIMENTS

The field of embodiments relates to human mobility apparatuses and, in particular, to improved crutch systems.

### BACKGROUND OF THE EMBODIMENTS

Standard crutches have been in use for many years, aiding individuals walk. However, long term use of standard crutches often results in arm pain (and pain elsewhere on the body of the user) due to the pressure place on the upper body of the user. Further, a user, using a standard crutch, often must keep the user's leg in a bent position so that it does not touch the ground. Additionally, standard crutches have no way of maintaining an upright position while in use without leaning on something, such as the arm of the user.

For at least these reasons, there are many negative aspect to using a standard crutch. An easier-to-use and more ergonomically designed crutch is thus needed.

Examples of related art are described below:

U.S. Pat. No. 6,783,555 generally describes a unilateral or bilateral orthotic or prosthetic device that facilitates reciprocating ambulation by a human user. The reciprocating gait orthosis includes a torso vest securely positionable around the torso of the user, at least one torso joint coupled to the torso vest such that the torso joint is approximately adjacent to the uses natural waist, and at least one hip member that couples the torso joint and torso vest to a conventional leg support assembly at the hip joint approximately adjacent the user's natural hip joint. The leg support assembly is adapted to couple to the user's leg or to replace the human user's missing leg to provide the user stable support when the user is in an upright position.

U.S. Pat. No. 4,884,587 generally describes an auxiliary apparatus designed and structured to be appended to the lower part of a cane or crutch so that the user may have additional mechanical means to help lift his leg or foot with the aid of the crutch or cane. The device comprises in general an L-shaped frame member, the lower leg of which functions essentially as a stirrup upon which the foot can be placed, while the vertical is adapted, when so used, to be rested against the lower shank of the cane or crutch. An adjustable U-shaped collar is pivotally mounted to the upper leg of the L-shaped member serving to affix the L-shaped frame to the crutch shank. By pivotally mounting the L-shaped frame to the collar member, the L-shaped frame member can be pivotably moved to a position that is retracted upward out of the way when not in use.

U.S. Pat. No. 9,180,037 generally describes a leg brace that includes a frame and a stirrup coupled to a lower end of the frame for engaging a ground surface during use. The leg brace further includes a first attachment assembly coupled to an upper end of the frame that is configured to removably secure the frame to a first portion of a user's leg and a second attachment assembly coupled to a lower end of the frame that is configured to removably secure the frame to a second portion of a user's leg.

British Patent Publication No. GB675275A generally describes various crutches. The lower part of a crutch is bent or cranked rearwardly in relation to the axis of the crutch. In the lefthand crutch shown, the greater part of its length is straight but the lower part is bent backwards at a small angle. The foot may be of arcuate form in the plane perpendicular to the direction of motion and the upper rest is preferably of U-shape with the rear limb longer and broader than the front limb, both limbs being padded. Alternatively the two limbs may be resilient and adjustable. The tubular member carrying the hand grip may be adjustable for height on the crutch stick or the stick may be divided with the lower section secured to the hand grip member and the upper section telescopically slidable in it, so as to provide a crutch of adjustable length. If desired the crutch may be bowed outwardly at the point 3a and a foot rest 4b may be attached to the foot member.

Japanese Patent Publication No. JP3728248B2 generally describes a crutch having an extension stick in the lower end portion of an arch body of a side arch equipped with an arm piece part and a grip part, the extension stick being configured to be extended/contracted relatively to the arch body of the side arch and the crutch is provided with an extension stick elongating/contracting device for extending the extension stick relative to the arch body of the side arch, an extension stick operating part for sending a signal for elongating the extension stick relatively to the arch body of the side arch, a signal for contracting the extension stick relatively to the arch body of the side arch and a signal for fixing the extension stick relatively to the arch body of the side arch to the extension stick elongating/contracting device, a stirrup hung down from the arch body of the side arch vertically movably for placing the foot of a user, a stirrup elevating device for elevating the stirrup, and a stirrup operating part for sending a signal for moving up the stirrup and a signal for holding the hanging height of the stirrup to the elevating device.

None of the art described above addresses all of the issues that the present invention does.

### SUMMARY OF THE EMBODIMENTS

According to an aspect of the present invention, a leg brace configured to be coupled to a crutch is provided. The leg brace includes an adjustable height portion, a leg receiving portion, and a securing mechanism configured to secure a portion of a leg of a user to the leg receiving portion.

According to another aspect of the present invention, a system for securing a leg to a crutch is provided. The system includes a crutch and a leg brace coupled to the crutch. The leg brace includes an adjustable height portion a leg receiving portion, and a securing mechanism configured to secure a portion of a leg of a user to the leg receiving portion.

It is an object of the present invention to provide the leg brace, wherein the leg brace further includes a coupling mechanism configured to couple the leg brace to a portion of a crutch.

It is an object of the present invention to provide the leg brace, wherein the coupling mechanism is an adjustable height mechanism.

It is an object of the present invention to provide the leg brace, wherein the securing mechanism is selected from the group consisting of one or more straps, one or more clips, one or more buttons, and one or more hook and loop fasteners.

It is an object of the present invention to provide the leg brace, wherein the leg receiving portion includes one or more materials selected from the group consisting of metal, rubber, and plastic.

It is an object of the present invention to provide the leg brace, wherein the leg receiving portion is configured to complimentarily match a shape of the leg of the user.

It is an object of the present invention to provide the leg brace, wherein the leg receiving portion is configured to receiving a portion of the leg above and below a knee of the user.

It is an object of the present invention to provide the leg brace, wherein the securing mechanism is located above the knee of the user.

It is an object of the present invention to provide the leg brace, wherein the leg brace includes a plurality of securing mechanisms..

It is an object of the present invention to provide the system for securing a leg to a crutch, wherein the leg brace is removably coupled to the crutch.

It is an object of the present invention to provide the system for securing a leg to a crutch, wherein the leg brace is permanently coupled to the crutch.

It is an object of the present invention to provide the system for securing a leg to a crutch, wherein the leg brace further includes a coupling mechanism configured to couple the leg brace to a portion of a crutch.

It is an object of the present invention to provide the system for securing a leg to a crutch, wherein the coupling mechanism is an adjustable height mechanism.

It is an object of the present invention to provide the system for securing a leg to a crutch, wherein the securing mechanism is selected from the group consisting of one or more straps, one or more clips, one or more buttons, and one or more hook and loop fasteners.

It is an object of the present invention to provide the system for securing a leg to a crutch, wherein the leg receiving portion includes one or more materials selected from the group consisting of metal, rubber, and plastic.

It is an object of the present invention to provide the system for securing a leg to a crutch, wherein the leg receiving portion is configured to complimentarily match a shape of the leg of the user.

It is an object of the present invention to provide the system for securing a leg to a crutch, wherein the leg receiving portion is configured to receiving a portion of the leg above and below a knee of the user.

It is an object of the present invention to provide the system for securing a leg to a crutch, wherein the securing mechanism is located above the knee of the user.

It is an object of the present invention to provide the system for securing a leg to a crutch, wherein the leg brace is configured to collapse against the crutch.

### BRIEF DESCRIPTION OF THE DRAWINGS

FIG. 1 shows a perspective view of a crutch system, according to an embodiment of the present invention.

FIG. 2 shows a side view of a crutch system according to an embodiment of the present invention.

FIG. 3 shows a front view of a crutch system, according to an embodiment of the present invention.

FIG. 4 shows a front vie of a leg brace portion, according to a first embodiment of the present invention.

FIG. 5 shows a front view of a leg brace portion, according to a second embodiment of the present invention.

FIG. 6 shows a front view of a leg brace portion, according to a third embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

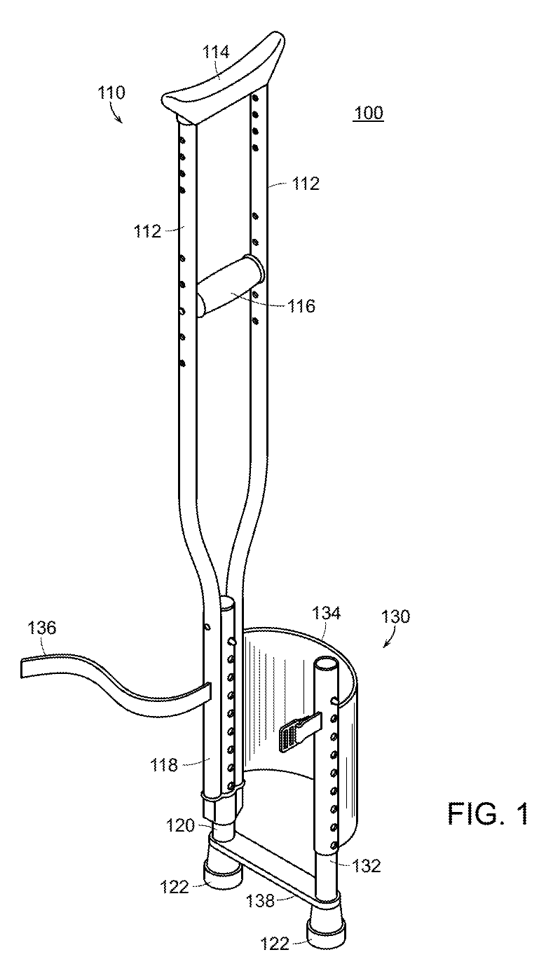

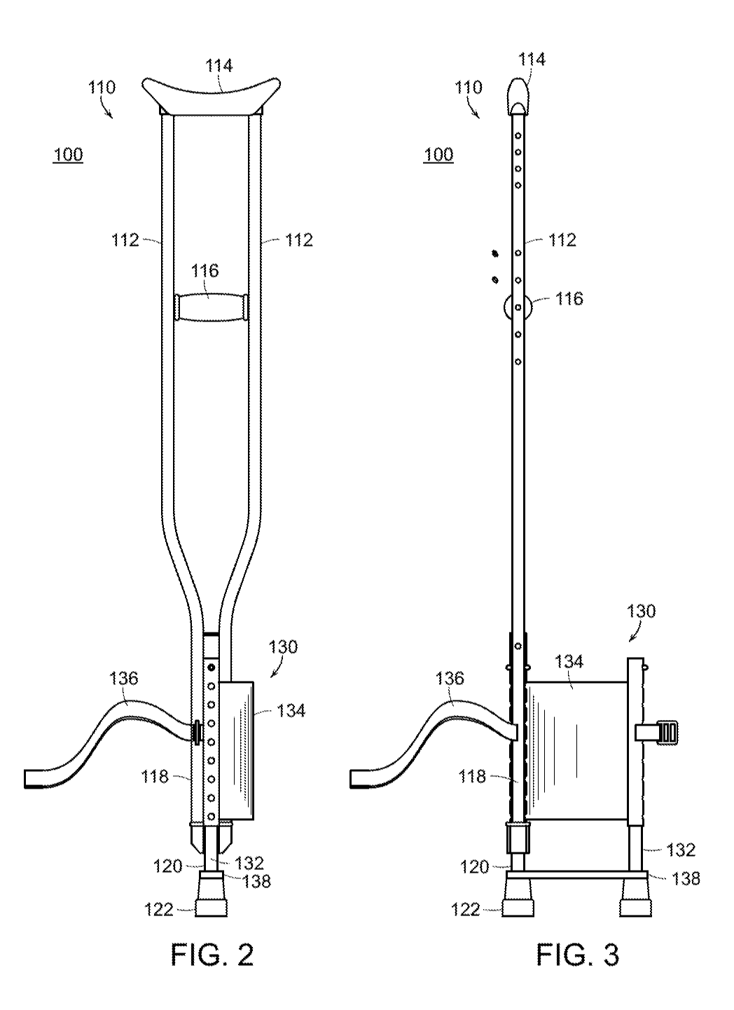

Referring, now to FIGS. 1-3, a perspective view (FIG. 1), a side view (FIG. 2), and a front view (FIG. 3) of a crutch system 100 are illustratively provided, in accordance with various embodiments of the present invention.

According to an embodiment, the system 100 includes a crutch apparatus 110, including two outer frame portions 112, a shoulder rest 114, a band rest 116, a leg height adjustment portion 118, a leg portion 120, and a stopper portion 122. According to an embodiment, one or more of the shoulder rest 114 and the hand rest 116 can be adjusted in regards to their positions on the frame portions 112.

The system 100 farther includes a leg brace portion 130 coupled to the crutch apparatus 110. According to an embodiment, the leg brace portion 130 is permanently coupled to the crutch apparatus 110. According to an embodiment, the leg brace portion 130 further includes a footrest 138 configured to enable a user to rest his/her foot, thereby elevating the leg of the user. According to an embodiment, the leg brace portion 130 is removably coupled to the crutch apparatus 110. According to an embodiment, the leg brace portion 130 is configured such that it is removable from the frame portions while the leg brace portion 130 is secured to the leg of the user. According to an embodiment, the footrest 138 is rigid. According to another embodiment, the footrest 138 is flexible. According to an embodiment, the system 100 includes a plurality of stoppers 122. According to an embodiment, the plurality of stoppers 122 may be located at any relevant position, such as on either side of the footrest 138, as shown in FIG. 1. According to an embodiment, the system 100 includes one stopper 122. According to an embodiment, the singular stopper 122 is positioned underneath the footrest 138. According to an embodiment, the singular stopper 122 is positioned below approximately a center portion of the footrest 138.

According to an embodiment, the system 100 is configured to secure a medical boot and/or other medical apparatus (e.g., cast, brace, etc.) to the crutch portion 110, with or without the leg brace portion 130. According to an embodiment, the leg brace portion 130 is configured to adequately house and/or secure the medical boot and/or other medical apparatus. According to an embodiment, the leg receiving portion 134 is configured to secure a medical boot and/or other medical apparatus (e.g., cast, brace, etc.). According to an embodiment, the leg brace portion 130 includes an adjustable height mechanism 132. Coupled to the adjustable height mechanism 132 is a leg receiving portion 134 configured to support part of a rear portion of a user's leg. According to an embodiment, the leg receiving portion 134 is curved. The leg receiving portion 134 may include metal, rubber, plastic, and/or any other suitable material, while maintaining the spirit of the present invention. According to an embodiment, the leg receiving portion 134 is configured to complimentarily match the shape of the leg of the user. According to an embodiment, the leg receiving portion 134 covers the back of part of the user's leg. According to an embodiment, the leg receiving portion 134 covers the entirety of the back of the user's leg. According to an embodiment, the leg receiving portion 134 includes padding such as, e.g., rubber, foam, cloth, and/or any other suitable padding materials.

According to an embodiment, the leg brace portion 130 includes a securing mechanism 136 for securing a leg of a user to the leg brace portion 130. The securing mechanism 136 may include one or more straps, one or more clips, one or more buttons, one or more hook and loop fasteners, and/or any other suitable securing mechanisms, while maintaining the spirit of the present invention. According to an embodiment, the securing mechanism 136 is flexible. According to an embodiment, the securing mechanism 136 is rigid. According to an embodiment, the securing mechanism 136 may include a rigid cover portion configured to be secured opposite the leg receiving portion 134, enabling the leg to be secured between the leg receiving portion 134 and the securing mechanism 136. According to an embodiment, the rigid cover portion may be removable. According to an embodiment, the securing mechanism 136 secures the leg such that the system 100 maintains its upright position, even when not held by the arm of the user.

According to an embodiment, the leg brace portion 130 is configured to house either a left leg or a right leg. According to an embodiment, the leg brace portion 130 is configured such that the leg receiving portion 134 can be repositioned and/or flexed to interchangeably receive a left leg and a right leg. According to an embodiment, the crutch apparatus 110 is coupled to the leg receiving portion 134 using a securing mechanism such as, e.g., a bolt, push buttons, straps, and/or any other suitable securing mechanism.

According to an embodiment, the leg receiving portion 134 includes a rigid material such as, e.g., steel, aluminum, carbon fiber, and/or any other suitable rigid materials. According to an embodiment, the leg receiving portion 134 in hides a flexible material such as, e.g., rubber, plastic, cloth, and/or any other suitable flexible materials. According to an embodiment, the leg receiving portion includes fabric, carbon fiber, and/or any other suitable material. According to an embodiment, the leg brace portion 130 is collapsible, enabling it to be pushed against the crutch apparatus 110, reducing the surface area of the system 100.

There are common problems that arise with the use of standard crutches. These problems include, but are not limited to, constantly having to hold the crutch upright with one or more arms, and arm and shoulder pain from the pressure exerted upon these areas from repeated use of the crutch. These issues can often be quite painful. According to an embodiment, the adjustable height mechanism 132 on the leg brace portion 130 is adjusted to level the leg receiving portion 134 prior to securement of the leg of the user. Once the leg of the user is secured to the leg receiving portion 134, using the securing mechanism 136, the crutch system 100 is capable of staying upright without the use of the user's arms. This enables more mobility for the user. Furthermore, the securing of the leg to the leg brace portion 130 may decrease the pressure and strain put on the arm of the user. According to an embodiment, the present invention relieves pressure in approximately the 15-30 lbs range as opposed to a regular crutch.

According to an embodiment, the outer frame portions 112 are collapsible and/or bendable, enabling a user to fit the crutch apparatus 110 under a table or other relevant object.

According to an embodiment, the crutch portion 110 is configured to house one or more objects (such as, e.g., a water bottle, an air tank, and/or any other suitable object) for storage and to ease transportation of the one or more objects.

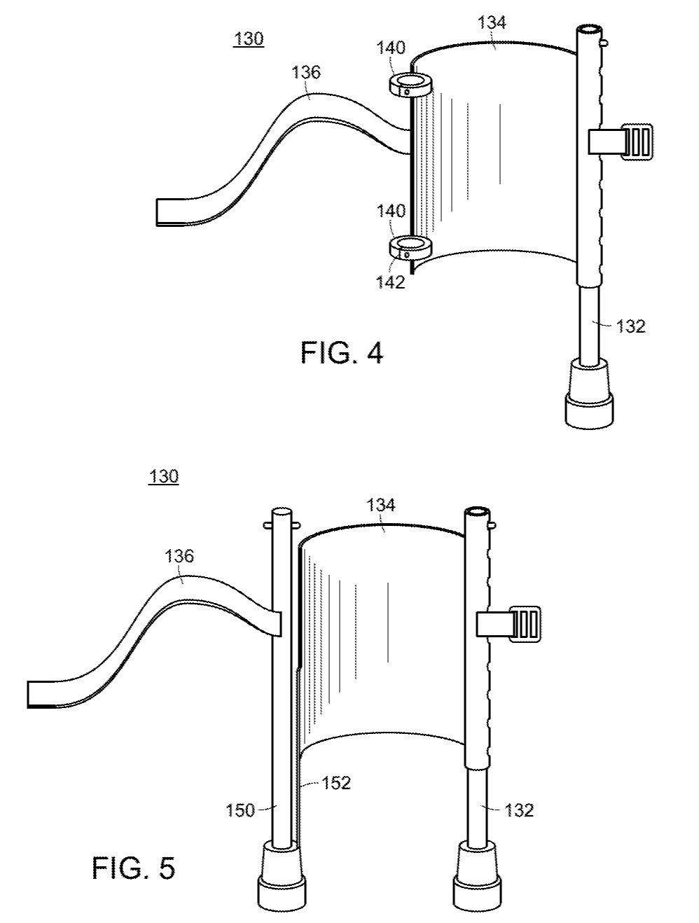

Referring now to FIG. 4, a front view of a leg brace portion 130 is illustratively depicted, in accordance with an embodiment of the present invention.

According to the embodiment shown in FIG. 4, the leg brace portion 130 is removably coupled to the crutch apparatus 110. The leg brace portion 130 shown in FIG. 4 has two or more hoops 140 for insertion of the leg portion 120 of the crutch apparatus 110, enabling the leg brace portion 130 to be removably coupled to the crutch apparatus 110. According to an embodiment, the hoops each include one or more rotatable clips 142 for attachment and removal from the crutch apparatus 110. According to an embodiment, the leg brace portion 130 is configured to be coupled to a crutch portion 110. However, it is noted that the leg brace portion 130 may be configured to be secured to any other suitable medical aid.

Referring now to FIG. 5, a front view of a leg brace portion 130 is illustratively depicted, in accordance with an embodiment of the present invention.

According to the embodiment shown in FIG. 5, the leg brace portion 130 includes an adjustable height apparatus 150 for insertion into the crutch apparatus 110. The adjustable height apparatus 150 is coupled to the leg receiving portion 134 via one or more connecting members 152. According to the embodiment shown in FIG. 5, the leg brace portion 130 is coupled to the crutch apparatus 110 by insertion of the adjustable height apparatus 150 into the crutch apparatus 110.

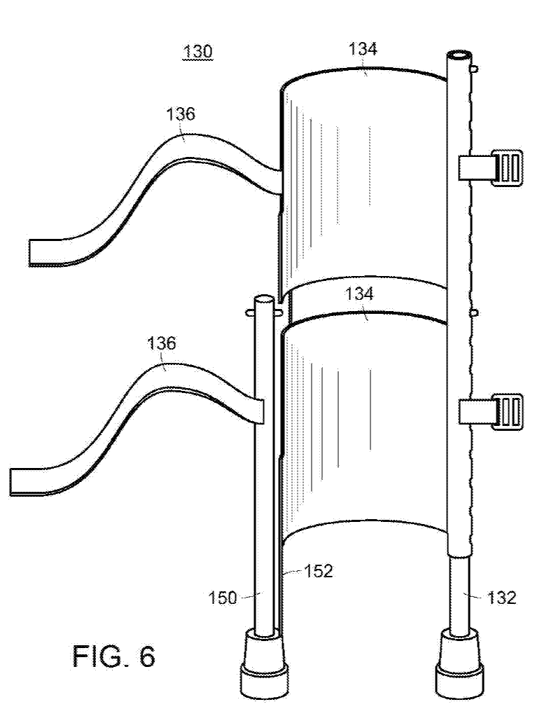

Referring now to FIG. 6, a front view of a leg brace portion 130 is illustratively depicted, in accordance with an embodiment of the present invention.

According to the embodiment shown in FIG. 6, the leg brace portion 130 includes two leg receiving portions 134 configured to secure the leg above and below the knee. According to an embodiment, the leg brace portion 130 may include multiple leg receiving portions 134 or may include one leg receiving portion 134 configured to secure the leg both above and below the knee. According to an embodiment, the leg receiving portion 134 may be above and/or below the knee of the user. According to an embodiment, the leg brace portion 130 may include one or more securing mechanisms above and/or below the knee of the user.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A leg brace configured to be coupled to a crutch, the leg brace comprising:
an adjustable height portion;
a leg receiving portion; and
a securing mechanism configured to secure a portion of a leg of a user to the leg receiving portion.

2. The leg brace as recited in claim 1, further comprising:
a coupling mechanism configured to couple the leg brace to a portion of a crutch.

3. The leg brace as recited in claim 2, wherein the coupling mechanism is an adjustable: height mechanism.

4. The leg brace as recited in claim 1, wherein the securing mechanism is selected from the group consisting of
one or more straps;
one or more clips;
one or more buttons; and
one or more hook and loop fasteners.

5. The leg brace as recited in claim 1, wherein the leg receiving portion includes one or more materials selected from the group consisting of:
metal;
rubber; and
plastic.

6. The leg brace as recited in claim 1, wherein the leg receiving portion is configured to complimentarily match a shape of the leg of the user.

7. The leg brace as recited in claim 1, wherein the leg receiving portion is configured to receiving a portion of the leg above and below a knee of the user.

8. The leg brace as recited in claim 7, wherein the securing mechanism is located above the knee of the user.

9. The leg brace as recited in claim 7, wherein the leg brace includes a plurality of securing mechanisms.

10. A system for securing a leg to a crutch, the system comprising:
a crutch; and
a leg brace coupled to the crutch, the leg brace including:
an adjustable height portion;
a leg receiving portion; and
a securing mechanism configured to secure a portion of a leg of a user to the leg receiving portion.

11. The system as recited in claim 10, wherein the leg brace is removably coupled to the crutch.

12. The system as recited in claim 10, wherein the leg brace is permanently coupled to the crutch.

13. The system as recited in claim 10, wherein the leg brace thither includes:
a coupling mechanism configured to couple the leg brace to a portion of a crutch.

14. The system as recited in claim 13, wherein the coupling mechanism is an adjustable height mechanism.

15. The system as recited in claim 10, wherein the securing mechanism is selected from the group consisting of:
one or more straps;
one or more clips;
one or more buttons; and
one or more hook and loop fasteners.

16. The system as recited in claim 10, wherein the leg receiving portion includes one or more materials selected from the group consisting of
metal;
rubber; and
plastic.

17. The system as recited in claim 10, wherein the leg receiving portion is configured to complimentarily match a shape of the leg of the user.

18. The system as recited in claim 10, wherein the leg receiving portion is configured to receiving a portion of the leg above and below a knee of the user.

19. The system as recited in claim 18, wherein the securing mechanism is located above the knee of the user.

20. The system as recited in claim 10, wherein the leg brace is configured to collapse against the crutch.
